(19) United States
(12) Patent Application Publication
(10) Pub. No.: US 2020/0087723 A1
(43) Pub. Date: Mar. 19, 2020

(54) METHODS AND SYSTEMS FOR DETERMINING PARALOGS

(71) Applicants: Illumina, Inc., San Diego, CA (US); Illumina Cambridge Limited, Nr Saffron Walden, Essex (GB)

(72) Inventors: Aaron L. Halpern, San Carlos, CA (US); Semyon Kruglyak, San DIego, CA (US); Peter Krusche, Basel (CH)

(21) Appl. No.: 16/467,930

(22) PCT Filed: Dec. 14, 2017
(86) PCT No.: PCT/US2017/066498

§ 371 Date: Jun. 7, 2019

Related U.S. Application Data
(60) Provisional application No. 62/434,876, filed on Dec. 15, 2016.

(51) Int. Cl. C12Q 1/6869; C12Q 1/6883

(52) U.S. Cl. CPC C12Q 1/6869; C12Q 2600/156; C12Q 1/6883

(57) ABSTRACT

Disclosed herein are systems and methods for spinal muscular atrophy (SMA) diagnosis from whole genome sequencing data. In one embodiment, a method comprises aligning whole genome sequencing (WGS) reads of a subject's sample to a modified reference sequence such as a modified reference genome sequence. After counting the reads supporting quasi-alleles at select positions of the reference sequence, the method can adjust for coverage and determine a number of functional SMN1 gene copies. The method can determine affected or carrier status of the subject based on the copy number of functional SMN1 gene copies.

### RELATED APPLICATIONS

The present application claims priority to U.S. Provisional Application No. 62/434876, filed on Dec. 15, 2016; the content of which is herein expressly incorporated by reference in its entirety.

### BACKGROUND

### Field

The present disclosure generally relates to the field of disease diagnosis and more particularly to determining the affected or carrier status for diseases such as spinal muscular atrophy caused by defective genes with highly similar paralogs using whole genome sequencing data.

### Description of the Related Art

Motor neuron diseases (MNDs) are a group of progressive neurological disorders that destroy motor neurons, the cells that control essential voluntary muscle activity such as speaking, walking, breathing, and swallowing. Normally, messages from motor nerve cells in the brain (called upper motor neurons) are transmitted to motor nerve cells in the brain stem and spinal cord (called lower motor neurons), and messages from the lower motor neurons are transmitted to particular muscles. Upper motor neurons direct the lower motor neurons to produce movements such as walking or chewing. Lower motor neurons control movement in the arms, legs, chest, face, throat, and tongue. Spinal motor neurons are also called anterior horn cells.

Spinal muscular atrophy (SMA) is an autosomal recessive, neuromuscular disorder characterized by loss of motor neurons and progressive muscle wasting, often leading to early death. The disorder is caused by a genetic defect in the SMN1 gene, which encodes survival of motor neuron (SMN) protein, a protein expressed in all eukaryotic cells and necessary for the survival of motor neurons. Lower levels of the protein result in loss of function of neuronal cells in the anterior horn of the spinal cord and subsequent system-wide muscle wasting (atrophy).

A person is affected with SMA if the person only has defective copies of the SMN1 gene. A person is a carrier of SMA if the person has one chromosome containing at least one normal copy of the SMN1 gene and at least one chromosome containing no normal copies of the SMN1 gene (i.e., either no copies of SMN1 or only defective copies of SMN1).

A small amount of SMN protein can be produced from a gene similar to SMN1 called SMN2. Several different versions of the SMN protein are produced from the SMN2 gene, but only one version (called isoform d) is full size and fully functional. The other versions are smaller and may be easily broken down. The full-size protein made from the SMN2 gene is identical to the protein made from SMN1; however, much less full-size SMN protein is produced from the SMN2 gene compared with the SMN1 gene. SMN1 and SMN2 genes are nearly identical and encode the same protein. The sequence difference between the two is a single nucleotide in exon 7 which is thought to be an exon splice enhancer. It is thought that gene conversion events may involve the two genes, leading to exchanges of sequence between SMN1 and SMN2.

### SUMMARY

Disclosed herein are systems and methods for diagnosing a disease based on mutations in non-unique portions of the genome. The systems and methods can be used to determine affected or carrier status for indications such as spinal muscular atrophy (SMA). In one embodiment, the systems and methods use whole genome sequencing (WGS) data to determine affected or carrier status. In one embodiment, a method can include: aligning WGS reads to a modified reference genome sequence; counting reads supporting quasi-alleles at select positions of the reference sequence, and adjusting for coverage and determining the number of functional SMN1 gene copies. The modified reference genome sequence can be a version of the reference genome sequence that has bases of SMN2 converted to a string of Ns of equal length (also referred to as SMN2-depleted reference genome sequence). The method can further include: determining WGS reads that include known inactivating mutations in an SMN1 gene. The method can further include: counting reads supporting other quasi-alleles at select positions; adjusting for coverage; and determining the number of copies of the SMN2 gene. The methods described herein may extend to diagnosis based on mutations in other non-unique portions of the genome.

In some embodiments, a system comprises a hardware processor configured to execute computer-executable instructions to perform any of the methods disclosed herein; and a data store configured to store whole genome sequencing data or diagnosis results. In some embodiments, a computer readable medium comprises a software program that comprises logic or instructions for performing any of the methods disclosed herein.

### BRIEF DESCRIPTION OF THE DRAWINGS

FIG. 1 is a flow diagram depicting an illustrative method for aligning whole genome sequencing read data to an SMN2-depleted reference genome for spinal muscular atrophy diagnosis.

FIG. 2 is a schematic illustration of the relationships between the inputs and outputs used to generate WGS reads derived from SMN1 or SMN2 aligned to SMN1 in FIG. 1.

FIG. 3 is a flow diagram depicting an illustrative method for using the whole genome sequencing read data aligned to the SMN2-depleted reference genome sequence in FIG. 1.

FIGS. 4A-4C schematically illustrate the relationships between the inputs and outputs used for spinal muscular atrophy diagnosis in FIG. 3.

FIGS. 5A and 5B schematically illustrate a graph-based method of variant calling, such as distinguishing single nucleotide polymorphisms (FIG. 5A), structural variants (FIG. 5B), and paralogs (FIG. 5C).

FIG. 6 is a flow diagram depicting an illustrative graph-based method of determining SMA status.

FIG. 7 depicts a general architecture of an example computing device configured to perform diagnosis of spinal muscular atrophy from whole genome sequencing data.

FIG. 8 is an exemplary plot of the sum of read counts supporting SMN2 vs. the sum of read counts supporting SMN1, which can be used to determine SMN1- and SMN2-specific copy numbers.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety concerning the related technology.

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

### Overview

Disclosed herein are systems and methods for diagnosing a disease based on mutations in non-unique portions of the genome. The systems and methods can be used to determine affected or carrier status of a subject for SMA using whole genome sequencing (WGS) data. A subject is affected with SMA if the subject has only defective copies of the SMN1 gene. A subject is a carrier of SMA if the subject has at least one chromosome containing at least one normal copy of the SMN1 gene and at least one chromosome containing no normal copies of SMN1 (i.e. either no copies of SMN1 or only defective copies of SMN1).

In one embodiment the genetic status of a subject may be determined by aligning WGS reads to a modified reference sequence. The modified reference sequence may include the SMN1 reference sequence (chr5, 70220767-70248842 on human genome reference sequence hg19 or GRCh37). The modified genome sequence may have bases of the SMN2 sequence (chr5, 69345350-69373422) converted to a string of Ns of equal length (also referred to as an SMN2-depleted or -masked reference genome sequence). The mapped WGS reads may then be counted to determine quasi-alleles at select positions of the modified reference sequence. “Quasi-alleles” refer to differences in sequences between the mapped WGS reads and the modified reference sequence. The differences may be due to polymorphisms of the SMN genes or due to differences between the SMN1 and SMN2 genes. An SMN gene refers to the SMN1 gene or the SMN2 gene, and the differences may be due to polymorphisms of either the SMN1 gene or the SMN2 gene. The select positions of the modified reference sequence can include positions of fixed differences between SMN1 and SMN2. The method may then adjust for coverage (the average read depth or the number of reads per unit length of the genome) and thereafter determine the number of functional SMN1 gene copies based on the number of reads that support quasi-alleles at select positions of the modified reference sequence counted. In some embodiments, the method can adjust for coverage by normalizing coverage depth (i.e. read count) by the genome-wide or chromosome-wide average for the sample being analyzed. Thus, the coverage is normalized against other regions of the genome for the same sample.

In other embodiments, the method may determine the WGS reads that contain known inactivating mutations of SMN1 by determining the sequences of the WGS reads at the known inactivating mutations. The method may also count the number of reads that support other quasi-alleles at the select positions. The method may then adjust for coverage and thereafter determine the number of copies of SMN2 based on the number of reads that support quasi-alleles at select positions of the modified reference sequence counted. The methods described here may extend to diagnosis based on mutations in other non-unique portions of the genome.

In some embodiments, the methods disclosed herein can be used to distinguish paralogs when paralogs (or paralogous exons) are similar enough in the genome reference sequence to make a read alignment ambiguous. For example, the paralogs can be SMN1/2, DUX4, RPS17, CYP2D6/7.

### Aligning Whole Genome Sequencing Read Data to a Modified Reference Genome

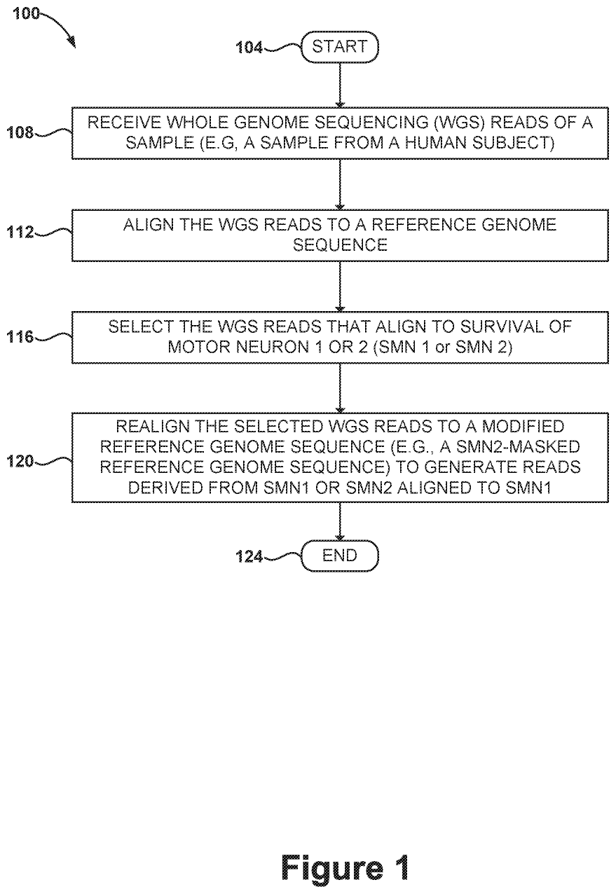

Spinal muscular atrophy (SMA) affected or carrier status can be determined from whole genome sequencing (WGS) read data. FIG. 1 is a flow diagram depicting an illustrative method 100 for aligning the WGS read data to a modified reference genome sequence, in particular an SMN2-depleted reference genome sequence. An SMN2-depleted reference genome sequence is a reference genome sequence with the sequence of SMN2 converted to a string of Ns of equal length. After beginning at start block 104, the method 100 proceeds to block 108. At block 108, the method 100 receives WGS read data of a sample. The sample can be from a subject such as a human subject. WGS is a laboratory process that determines the complete DNA sequence of an organism's genome at a single time, including the organism's chromosomal DNA as well as DNA contained in the mitochondria. Techniques for generating WGS includes sequencing techniques such as sequencing by synthesis using MINISEQ, MISEQ, NEXTSEQ, HISEQ, and NOVASEQ sequencing instruments from Illumina, Inc. (San Diego, Calif.).

From block 108, the method 100 proceeds to block 112, wherein the method 100 aligns the WGS reads to a reference genome sequence. The reference genome sequence of a human subject can be a human reference genome sequence such as the hg16, hg17, hg18, hg19, or hg38 reference human genome sequence (These reference human genome sequences are available from http://hgdownload.cse.ucsc.edu/downloads.html). Methods for aligning the WGS reads to a reference genome sequence can utilize aligners such as Burrows-Wheeler Aligner (BWA) and iSAAC. Other alignment methods include BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM.

The method 100 proceeds to block 116 from block 112 where the method 100 selects the WGS reads that are aligned to the portions of the reference genome sequence corresponding to either the SMN1 or SMN2 genes for further evaluation. A WGS read may be selected as corresponding to the SMN1 or SMN2 genes regardless of the confidence of the alignment. Alignment confidence can be represented by alignment confidence scores such as the MAPQ score.

From block 116, the method 100 proceeds to block 120. At block 120, the method 100 aligns the WGS reads selected at block 116 to a modified reference sequence (also referred to as realigning the WGS reads because the WGS reads are aligned to a modified reference sequence subsequent of the WGS reads are aligned to a reference sequence). The realignment of the WGS reads at block 120 generates reads derived from SMN1 or SMN2 aligned to SMN1. The modified reference sequence can be a version of the reference sequence used in block 112 with bases of SMN2 converted to a string of Ns of equal length. The modified reference sequence can be referred to as an SMN2-depleted reference sequence. The differences in sequences between the mapped WGS reads and the modified reference sequence can be referred to as “quasi-alleles.” The differences may be due to polymorphisms of the SMN genes or due to differences between the SMN1 and SMN2 genes. An SMN gene refers to the SMN1 gene or the SMN2 gene, and the differences may be due to polymorphisms of either the SMN1 gene or the SMN2 gene. The method 100 ends at block 124.

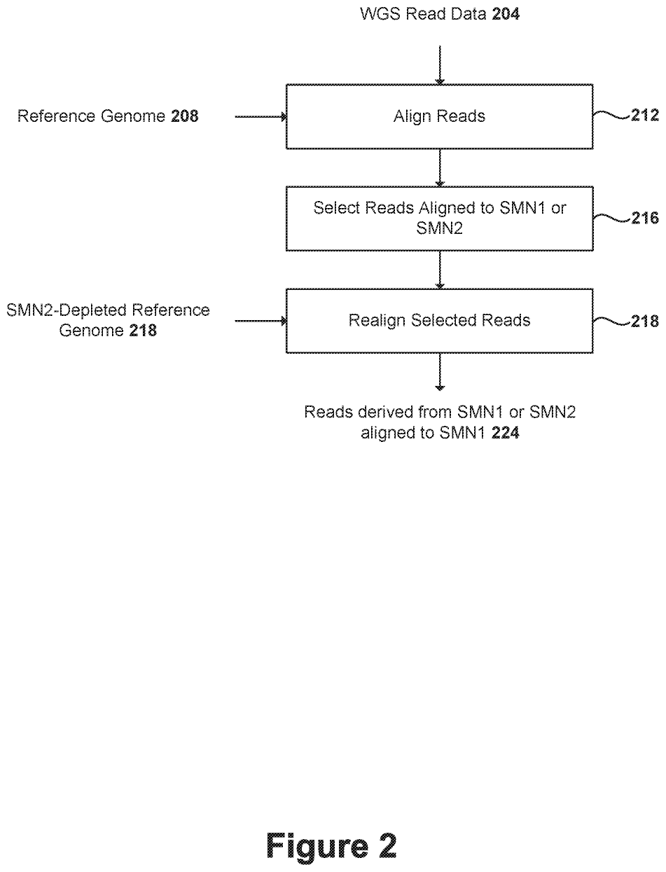

FIG. 2 is a schematic illustration of the relationships between the inputs and outputs used to generate WGS reads derived from SMN1 or SMN2 aligned to SMN1 in FIG. 1. WGS read data 204 comprising WGS reads are aligned to a reference genome sequence 208 at block 212. The WGS reads that are aligned to SMN1 or SMN2 in the reference genome sequence 208 can be selected at block 216 for realignment to an SMN2-depleted reference genome sequence 218 at block 220. The realignment at block 220 generates reads derived from SMN1 or SMN2 aligned to SMN1 224.

### Determining Spinal Muscular Atrophy Affected and Carrier Status

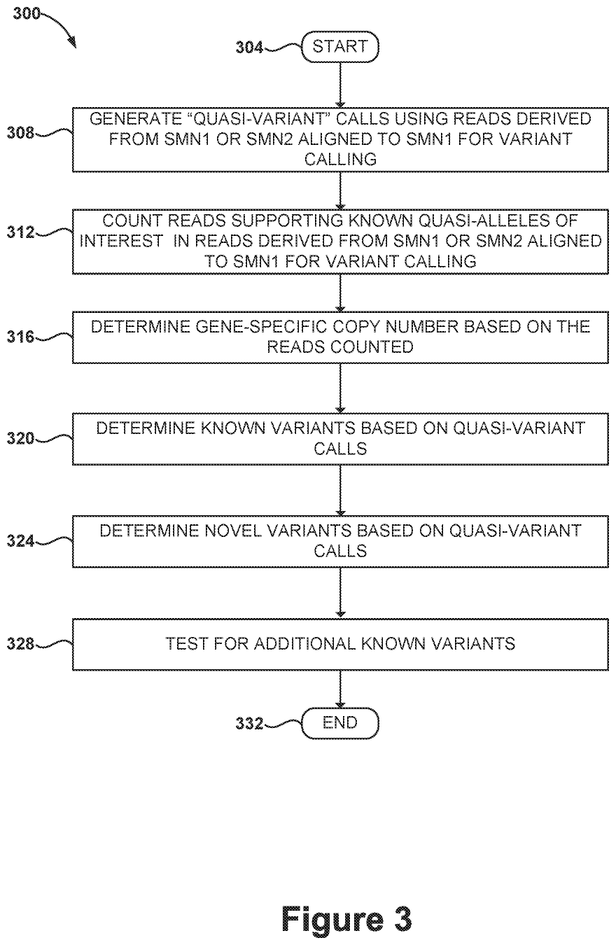

FIG. 3 is a flow diagram depicting an illustrative method 300 for using the whole genome sequencing read data aligned to the SMN2-depleted reference genome sequence in FIG. 1 for spinal muscular atrophy diagnosis. The illustrative method 300 may be implemented following implementation of method 100, discussed above, such that block 308 occurs subsequent to block 120 described above.

Reads that are aligned to SMN1 in block 120 can be used for determining copy numbers and possible variants in SMN1 and SMN2. For example, alignment of the WGS reads to the SMN2-depleted reference allows high confidence identification of reads derived from either SMN1 or SMN2. Thus, reads aligned to highly-repetitive portions of SMN1 with high confidence scores are not likely to be derived from other regions of the reference sequence. These realigned reads can be used to estimate the total number of copies of SMN1 and SMN2 in the subject genome, an SMN1-specific copy number and an SMN2-specific copy number. These realigned reads can also be used to estimate small variations between the SMN1 reference sequence and copies of SMN1 or SMN2 in the subject whose sequence is being analyzed. From this, several pieces of information can be obtained that are informative regarding SMA affected or carrier status.

The reads aligned to SMN1 on the SMN2-depleted reference can be further processed before diagnosis of SMA status.

After the method 300 begins at block 304, the method 300 generates “quasi-variant” calls using the reads derived from SMN1 or SMN2 aligned to SMN1 for variant calling at block 308. The quasi-variant calls show differences from the SMN1 reference sequence. Such quasi-variants can also show fixed differences between SMN1 and SMN2, polymorphisms, or mutations of either SMN1 or SMN2 in the sample.

A quasi-variant call is a determination that there exists a sequence in the sample being analyzed that is recognizably similar to the SMN1 reference sequence but differing from the SMN1 reference sequence in details. Whereas a standard variant call implies a change of the sequence at a specific location in the genome, a quasi-variant may imply one of three or more possibilities. These possibilities include a) a change to the sequence at the indicated location; b) a difference between the indicated location (in SMN1) and the corresponding piece of a highly similar region (SMN2); or c) a change relative to the reference at the highly similar region (SMN2). These three possibilities correspond to a variant in SMN1, a difference between SMN1 and SMN2, and a variant in SMN2. The phrase “quasi-variant,” rather than simply “variant,” indicates the ambiguity.

From block 308, the method 300 proceeds to block 312, where the method 300 counts the number of reads in the reads derived from SMN1 or SMN2 aligned to SMN1 supporting known quasi-alleles of interest using a reference of fixed differences between SMN1 and SMN2.

The method 300 proceeds to block 316 from block 312, where the method 300 determines a gene-specific (SMN1 or SMN2) copy number based on the number of reads counted at block 312. By comparing the reads derived from SMN1 or SMN2 aligned to SMN1 with fixed differences between SMN1 and SMN2, a copy number of SMN1 and a copy number of SMN2 can be determined.

The gene-specific copy number can, in turn, be used to identify affected or carrier status of a subject because the sizeable majority, approximately 95%, of SMA cases, and of carrier haplotypes, are due to one of two types of change that result in the absence of the SMN1 version of exon 7. This can result from the loss (total absence or quantitative depletion for affected and carrier respectively) of the SMN1 version of exon 7, or from gene conversion of exon 7 so that the sequence in SMN1 exon 7 matches the SMN2 reference. A subject is affected with SMA if the subject has only defective copies of the SMN1 gene. A subject is a carrier of SMA (but not affected by SMA) if the subject has at least one chromosome containing at least one normal copy of the SMN1 gene and at least one chromosome containing no normal copies of SMN1 (i.e. either no copies of SMN1 or only defective copies of SMN1).

The genetics of SMA and existing, non-whole genome sequencing methods for SMA molecular diagnosis have been described in Prior, T W, et al.: Technical standards and guidelines for spinal muscular atrophy testing, Genet Med. 2011 July, 13(7):686-94, the content of which is incorporated herein in its entirety. Briefly, there is a key single-base difference between functional SMN1 and SMN2 that falls in exon 7 of the canonical transcript of SMN1. The sizeable majority, approximately 95%, of SMA cases, and of carrier haplotypes, are due to one of two types of change that can be detected as the loss (total absence or quantitative depletion for affected and carrier respectively) of the SMN1 version of exon 7. One change is a deletion of all or part of SMN1 that includes exon 7. The second change is a gene conversion replacing a region including exon 7 of SMN1 with the homologous sequence from SMN2.

Affected status for most affected individuals can thus be detected as the absence, or near absence (to allow for one or more sequencing errors) of the quasi-allele matching the SMN1 reference base at a specific position of exon 7. This can be determined by examining the SMN2-depleted variant call results at the relevant position of SMN1 exon (a homozygous call for the SMN2-specific quasi-allele indicating SMA affected status) or by performing a test on the counts of reads supporting the relevant quasi-alleles. In some embodiments, performing the test on the counts of reads supporting the relevant quasi-alleles can include: if fewer than X reads matching the normal SMN1 sequence are observed, the sample is labeled as “affected.” If more than Y reads matching the normal SMN1 sequence are observed, the sample can be labeled as “unaffected.” The thresholds X and Y can be determined empirically. The thresholds X and Y can depend on the depth of coverage. Alternatively or in addition, the thresholds X and Y can be adjusted based on the desired or acceptable accuracy. In some embodiments, the desired or acceptable accuracy can be determined for boundary cases. In some embodiments, performing the test on the counts of reads supporting the relevant quasi-alleles can be based on probabilistic models. The probabilistic models can be generated based on one or more sequencing errors or haplotype sampling. In some embodiments, population- or family-based priors could be incorporated into these processes.

Carrier status can be identified for most carriers by a quantitative reduction of reads that can be attributed to SMN1 rather than SMN2. It may appear that any or all positions differing in the reference sequences of SMN1 and SMN2 could be used in identifying carrier status. However, empirical assessments have indicated that many such differences reflect errors in the reference sequences or uncommon variants in the individual whose DNA provided the sequence of the reference, rather than fixed differences between the paralogous copies. As such, positions differing from the reference sequences of SMN1 and SMN2 cannot reliably be used to assess an SMN1-specific copy number.

However, examination of a large collection of unaffected individuals described in Example 1 below did identify a number (>10) of quasi-variants near exon 7 that are quasi-heterozygous in virtually all samples, with quasi-alleles matching differences in the reference sequences of SMN1 and SMN2. Variants may not be quasi-heterozygous in all samples due to samples that have zero copies of SMN2, or possibly SMA-affected individuals, should such samples be expected in the cohort. Counts of reads that support the SMN1 quasi-alleles at these positions can be used to infer the number of intact SMN1 copies present in the sample. Similarly, an SMN2 copy number can be determined.

When determining a gene-specific copy number to determine affected or carrier status, the method 300 at block 316 may implement one or more methods for improving copy number calls. In some embodiments, the method 300 may adjust for coverage by normalizing coverage depth (i.e. read count) by the genome-wide or chromosome-wide average for the sample being analyzed. Thus, the coverage is normalized against other regions of the genome for the same sample. Other methods for improving copy number calls include GC correction, normalization against a group of control samples, or characterization of sequence uniqueness to improve the results. GC correction has been described in Benjamini, Y, et al., Summarizing and correcting the GC content bias in high-throughput sequencing, Nucl. Acids Res., 2012, 40 (10): e72, doi:10.1093/nar/gks001, and Miller, C A, et al., ReadDepth: A Parallel R Package for Detecting Copy Number Alterations from Short Sequencing Reads, PLoS One., 2011, 6: e16327. doi: 10.1371/journal.pone.0016327; the content of each is incorporated by reference in its entirety.

The method 300 proceeds from block 316 to block 320, where the method 300 determines known variants based on quasi-variant calls generated at block 308. Given a list of known variants and a set of quasi-variant calls, the quasi-variant calls can be labeled as matching (i.e. consistent with) or not matching (inconsistent with) known variants in the list. Not all affected individuals have zero SMN1-like exon 7, as there are other mutations that disrupt the function of SMN1. Approximately 5% of affected individuals have one haplotype that has lost or gene-converted exon 7 but other mutations on the other haplotype. A portion of these may be identified by the presence of specific, known mutations at block 320.

The method 300 proceeds from block 320 to block 324, wherein the method 300 determines novel variants based on quasi-variant calls generated at block 308. Given a list of known variants and a set of quasi-variant calls, the quasi-variant calls can be labeled as not matching (i.e. inconsistent with) known variants in the list. These quasi-variant calls that are labeled as not matching known variants can be novel variants. Approximately 5% of affected individuals have one haplotype that has lost or gene-converted exon 7 but other mutations on the other haplotype. A portion of these may have novel or previously uncharacterized mutations, which can be identified in the quasi-variants called as described above with reference to block 308.

The method 300 proceeds from block 324 to block 328. At block 328, the method 300 tests for additional known variants by searching for reads containing specific kmers or other methods of genotyping one or more prior variants. The method 300 can determine whether a match between specific known variants of interest and quasi-variant calls. Affected status may be determined as the result of compound heterozygosity if the SMN1-specific copy number is estimated as one and a known or novel disruptive (quasi-) variant is detected. In some embodiments, detection of known or novel variants can include the use of structural variant detection methods in addition to single nucleotide variant (SNV) or indel detection. Indel refers to the insertion or the deletion of bases in the genome. Detection of carriers containing known disruptive variants of SMN1 can be performed similarly. The method 300 ends at block 332.

One challenge to accurate carrier status testing is the existence of haplotypes containing two (intact) copies of SMN1. An individual with one such haplotype and another haplotype with no intact copies of SMN1 will be a carrier as the zero-copy haplotype may be passed on. As carrier status is largely detected as a copy number change, such individuals can typically receive a false negative result in a carrier screen using standard methods. The methods described here may be no more and no less subject to this limitation. The method 300 may implement one or more techniques for reducing the impact of this problem by detecting a known haplotype carrying two copies of SMN1. An example of such techniques is described in Luo, M, et al., An Ashkenazi Jewish SMN1 haplotype-specific to duplication alleles improves pan-ethnic carrier screening for spinal muscular atrophy, Genet Med 2014, 16:149-56, the content of which is incorporated herein in its entirety.

The methods described above may give an inaccurate answer. The copy number methods may be confounded by chance deviations from expected numbers of reads, or by gene conversions affecting only a subset of the SMN1/SMN2-distinguishing quasi-variants. Potentially disruptive quasi-variants may be attributed to SMN1 when in fact they belong to SMN2, or vice versa. These potential errors limit sensitivity and specificity of the testing, but they are not expected to be common, and equally affect accepted (non-NGS) methods for SMA testing.

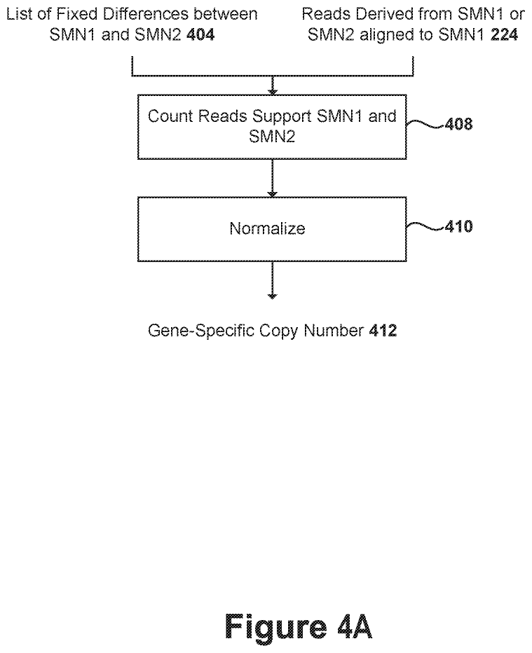

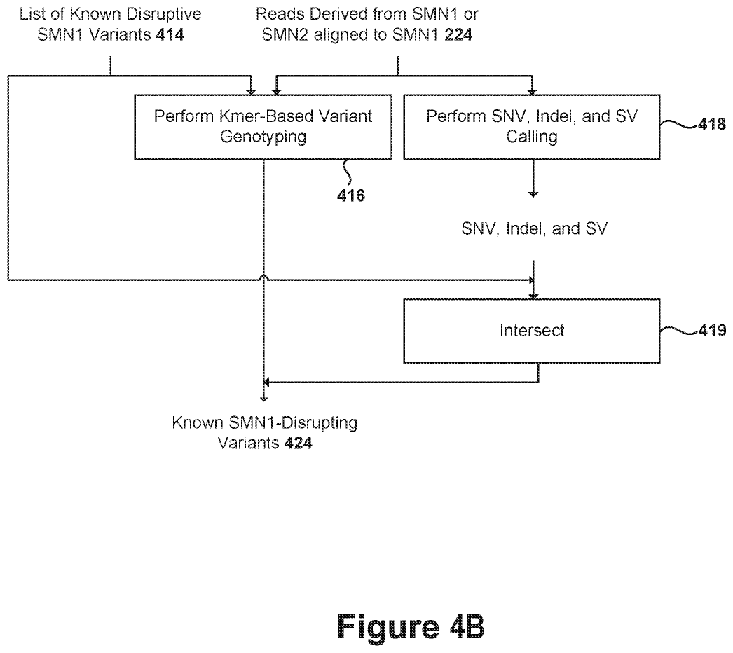

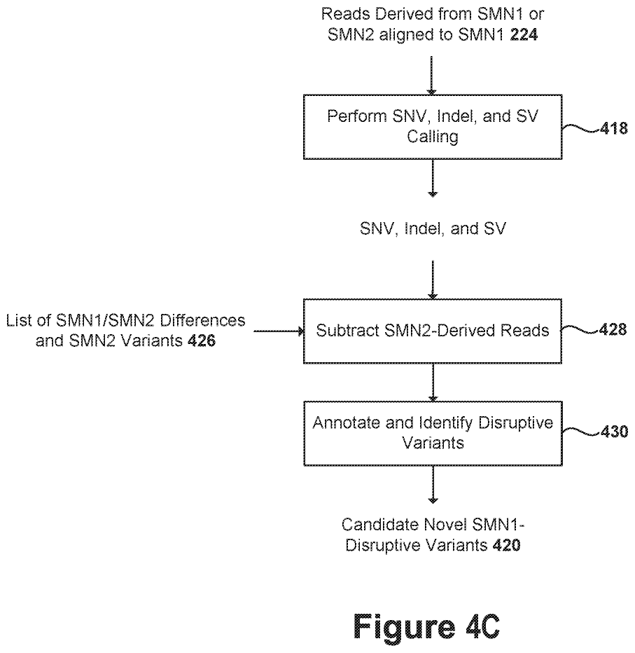

FIGS. 4A-4C schematically illustrate the relationships between the inputs and outputs used for spinal muscular atrophy diagnosis in FIG. 3. The reads derived from SMN1 or SMN2 aligned to SMN1 224 can be compared to a list of fixed differences between SMN1 and SMN1 404 to determine the number of reads in the reads derived from SMN1 or SMN2 aligned to SMN1 supporting known quasi-alleles of interest at block 408. After normalizing the number of reads supporting known quasi-alleles of interest at block 410, a gene-specific (SMN1 or SMN2) copy number is determined.

Reads derived from SMN1 or SMN2 aligned to SMN1 224 can be compared to a list of known disruptive SMN1 variants 414 using kmer-based variant genotyping at block 416 to test for additional known SMN1 variants. After detecting a single nucleotide variant (SNV), indel, or structural variant (SV) at block 418 using the reads derived from SMN1 or SMN2 aligned to SMN1 224, additional known SMN1 variants can be tested at block 424 by determining the intersection at block 419 of known disruptive SMN1 variants 414 and SNV or indel detected. SNVs and indels may be detected using tools or methods such as GATK, FreeBayes, Platypus, or Strelka. CNVs may be detected using tools or methods such as CANVAS, GenomeSTRIP, or CNVnator. SVs may be detected using tools or methods such as MANTA, BreakDancer, or Pindel.

SMN2-derived reads can be subtracted at block 428, based on a list of SMN1/SMN2 differences and SMN2 variants 426, from the SNV or indel detected at block 418. The resulting reads can be annotated to identify candidate novel SMN1-disrupting variants 420 at block 430.

### Graph-Based SMA Status Determination

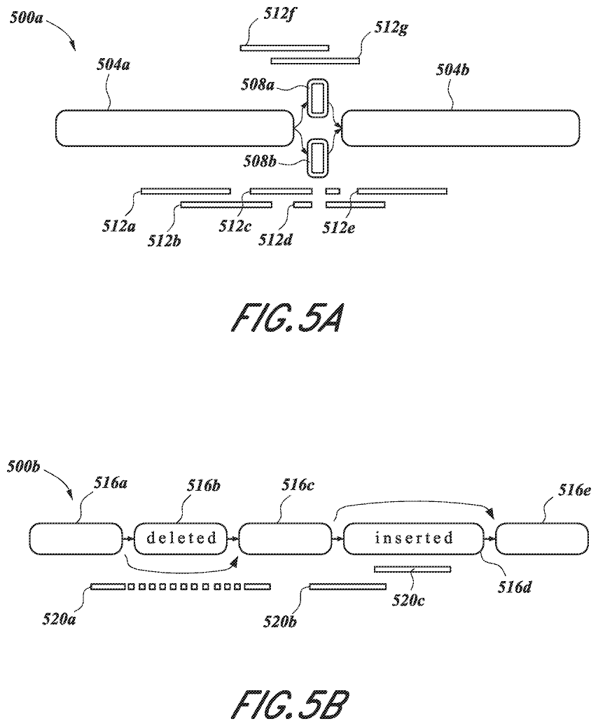

FIGS. 5A and 5B schematically illustrate a graph-based method of distinguishing paralogs, such as SMN1 and SMN2. The graph-based method can encode differences between paralogs and between variants of each paralog as different paths in the graph. A graph can represent a reference sequence of a first paralog, a reference sequence of a second paralog, and variants of each paralog. The method can be used to distinguish when paralogs (or paralogous exons) are similar enough in the genome reference sequence to make read alignment ambiguous, such as DUX4, RPS17, CYP2D6/7.

Referring to FIG. 5A, a graph 500a can include two non-branch nodes 504a, 504b and two branch nodes 508a, 508b connected by edges. The non-branch nodes 504a, 504b represent sequences of the paralogs that are invariant within each paralog and between the paralogs. For example, the non-branch nodes 504a, 504b can represent parts of the sequences of SMN1 and SMN2 that are invariant within SMN1, within SMN2, and between SMN1 and SMN2. The nodes 504a, 504b, 508a, 508b form two paths, 504a-508a-504b, 504a-508b-504b, that encode variants of a paralog, such as SMN1. The variants of a paralog can be a cytosine base or thymine base at position 873 in exon 7 of an SMN1 reference sequence, which corresponds to chromosome position 70247773 on chromosome 5. Chromosome position 70247773 on chromosome 5 in a reference sequence is a cytosine base. If that chromosome position has a thymine base instead, the resulting splice variant is translated into an inactive SMN1 protein. Sequence reads 512a-512g of a subject derived from the paralogs can be aligned to the graph 500a to determine the variant(s) the subject has. As illustrated in FIG. 5A, three of the seven sequence reads 512a, 512b, 512e can be aligned to the non-branch nodes 504a, 504b representing invariant sequences of the paralogs. Two of the seven sequence reads 512c, 512d can be aligned along a path containing nodes 504a, 508b, 504b representing one of the two variants. The remaining two of the seven sequence reads 512f, 512g can be aligned to a path containing the nodes 504a, 508a, 504b representing the other variant. Accordingly, the subject can be determined to have both the variants represented by the branch nodes 508a, 508b.

Referring to FIG. 5B, a graph 500b can include five non-branch nodes 516a-516c connected by edges. The edge connecting non-branch node 516a and non-branch node 516c represents a deletion of at least one nucleotide from the invariant sequences represented by the non-branch nodes 516a, 516c. The deleted sequence is represented by node 516b. The non-branch nodes 516a, 516-b, 516c form two paths, 516a-516b-516c representing the variant without the deletion and 516a-516c representing the variant with the deletion. Node 516d represents an inserted sequence of at least one nucleotide between the invariant sequences represented by nodes 516c, 516e, the edge connecting node 516c and node 516e represents an alternative where this insertion is not present. The nodes 516c, 516d, 516e form two paths, 516c-516e representing the variant without the insertion and 516c-516d-516e representing the variant with the insertion. In one embodiment, the insertion and deletion represented by the paths in graph 500b represent differences between two paralogs. The graph 500b thus encodes all four combinations representing variants with or without the deletion and with or without the insertion. For example, there is a common long deletion that removes a large portion of SMN1 (chr5:70244113-70250418) or SMN2 (chr5:69351655-69374999), including exon 7. Such deletions can be incorporated into the graph using edges between non-branch nodes.

As illustrated in FIG. 5B, one of the three sequence reads 520a can be aligned to the non-branch nodes 516a, 516c along edge 516a-516c representing the variant with the deletion. One of the sequence reads 520b can be aligned to the path containing non-branch node 516c and non-branch node 516d representing the variant with the insertion. The remaining sequence read 520c can be aligned to the non-branch node 516d representing variant with the insertion. Accordingly, the subject can be determined to have both the variants represented by the paths 516a-516c, 516c-516d-516e.

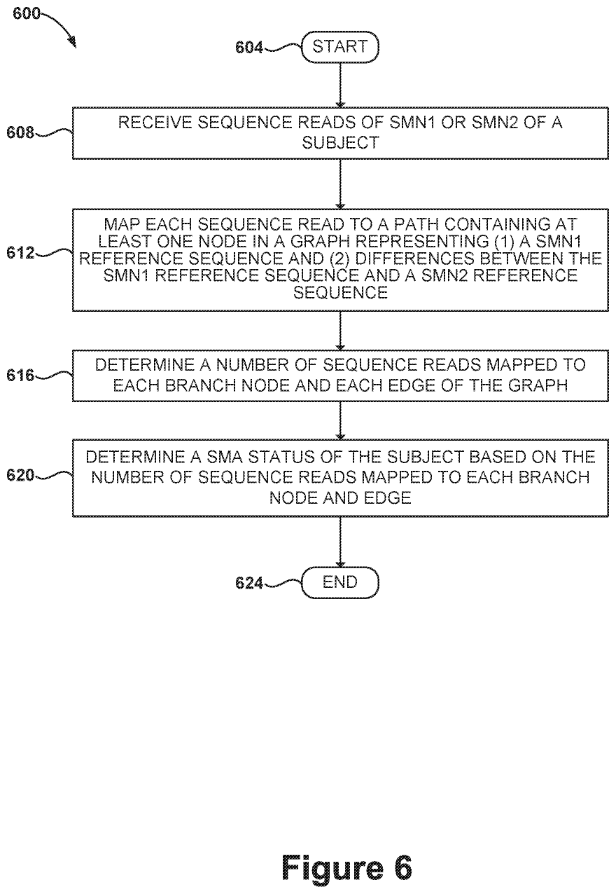

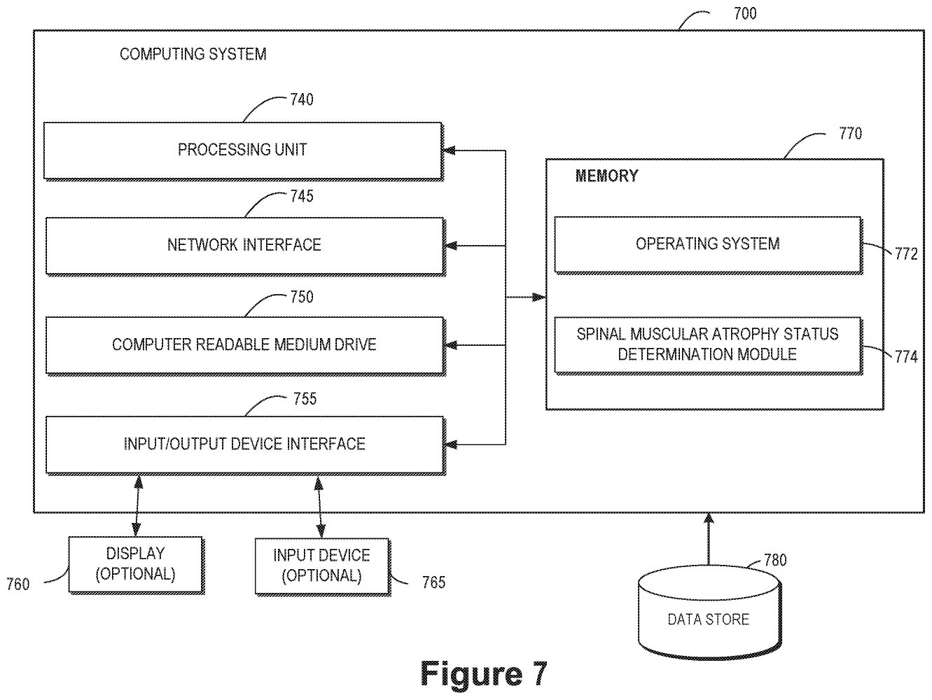

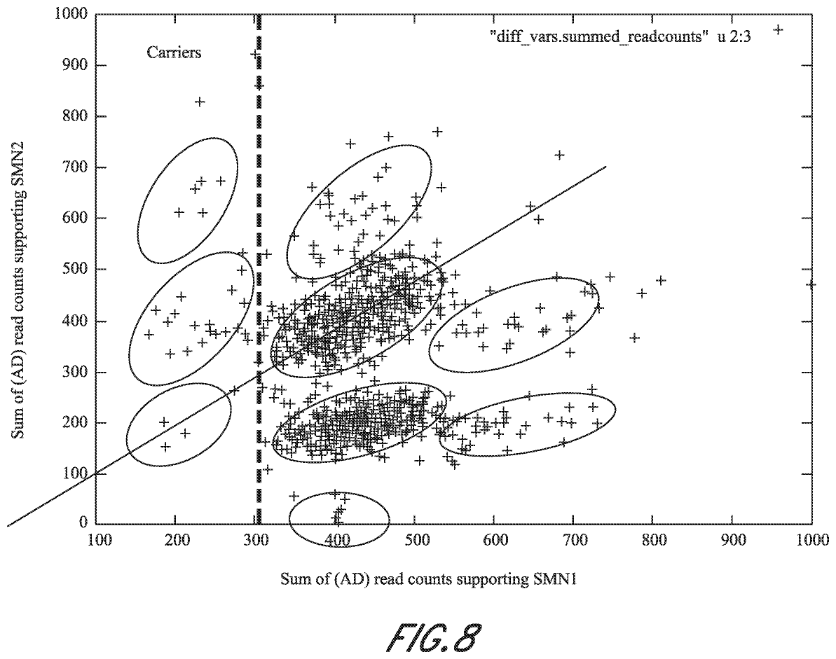

A graph-based method for distinguishing paralogs, such as SMN1 and SMN2, can be used for determining SMA status of a subject, including copy number estimation. FIG. 6 is a flow diagram depicting an illustrative graph-based method 600 for determining SMA status. After the method 600 begins at block 604, the method 600 proceeds to block 608, where a computing system, such as the computing device 700 described with reference to FIG. 7, receives a plurality of sequence reads of SMN1 or SMN2 of a subject.

The method 600 proceeds to block 612 from block 608, wherein the computing system maps each sequence read to a path containing at least one node in a graph representing an SMN1 reference sequence and differences between the SMN1 reference sequence and an SMN2 reference sequence. The graph includes multiple paths. Each path can be represented as an ordered list of one or more nodes of a plurality of branch nodes and non-branch nodes where an edge exists between each two subsequent nodes. By concatenating the sequences for these nodes in the listed order, the paths can represent a survival of motor neuron 1 (SMN1) reference sequence, sequence differences between the SMN1 reference sequence and a survival of motor neuron 2 (SMN2) reference sequence, variants of SMN1, and variants of SMN2. For example, known variants in SMN2 can be used to rule out treating these variants as possible disruptions of SMN1 and also to avoid overestimating the number of intact SMN2 copies.

The plurality of connected branch nodes and non-branch nodes can represent a graph with paths formed by the connected nodes encoding or representing the SMN1 reference sequence, differences between the SMN1 reference sequence and the SMN2 reference sequence, variants of SMN1, and variants of SMN2. The computing system may store the graph as a data structure for determining the SMA status of the subject. The computing system can generate the data structure representing the plurality of branch nodes and the plurality of non-branch nodes connected by the plurality of edges. The computing system can graphically display or cause display of a graph comprising the plurality of branch nodes and the plurality of non-branch nodes connected by the plurality of edges as a graph.

The plurality of non-branch nodes and a subset of the plurality of branch nodes connected by two orr more edges can represent the SMN1 reference sequence. With reference to FIG. 5A, the non-branch nodes 504a, 504b and the branch node 508a may present the SMN1 reference sequence. In one embodiment, two non-branch nodes connected to the same two non-branch nodes can represent a difference between the SMN1 reference sequence and the SMN2 reference sequence, a difference between the SMN1 reference sequence and a variant of SMN1, a difference between the SMN2 reference sequence and a variant of SMN2, or any combination thereof. For example, the branch nodes 508a, 508b in FIG. 5A connected to the same two non-branch nodes 504a, 504b can represent a difference between the SMN1 reference sequence and the SMN2 reference sequence. In another embodiment, one non-branch node connected to two non-branch nodes can represent an insertion of at least one nucleotide into the SMN1 reference sequence or a deletion of at least one nucleotide from the SMN1 reference sequence. With reference to FIG. 5B, one non-branch node 516c connected to two non-branch nodes 516a, 516b represent a deletion of the sequence represented by the non-branch node 516b from the SMN1 reference sequence. One non-branch node 516e connected to two non-branch nodes 516c, 516d can represent an insertion of the sequence represented by the non-branch node 516d into the SMN1 reference sequence.

Referring to FIG. 6, the method 600 proceeds to block 616 from block 612, wherein the computing system determines a number of sequence reads mapped to paths containing each branch node, non-branch node, and/or edges connecting two nodes. With reference to FIG. 5A, each sequence read 512a-512g can be mapped to one or more nodes 504a, 504b, 508a, 508b based on the sequence of the read and the sequences represented by the nodes 504a, 504b, 508a, 508b. With reference to FIG. 5B, each sequence read can be mapped to one or more nodes 516a-516e. In one embodiment, the alignment method determines an optimal local alignment to the graph and does not count read sequences for which multiple different optimal alignments exist in order to exclude reads which are not useful for disambiguating between paralog variants. An excluded read may be aligned to two or more paths with the same or similar alignment scores.

Referring to FIG. 6, the method 600 proceeds to block 620 from block 616, wherein the computing system determines a spinal muscular atrophy (SMA) status of the subject based on the number of sequence reads mapped to each of the plurality of branch nodes and edges. In one embodiment, determining the SMA status of the subject can include determining a number of sequence reads mapped to a node, such as the branch node 508a, representing a sequence difference between the SMN1 reference sequence and the SMN2 reference sequence. For example, the branch node 508a may represent a cytosine base at position 873 in exon 7 of the SMN1 reference sequence. The SMA status of the subject can be determined as the affected status if the number of sequence reads mapped to the branch node representing the SMN1 reference sequence is below a threshold. The SMA status of the subject can be determined as the carrier status or the unaffected status if the number of sequence reads mapped to the branch node representing the SMN1 reference sequence is not below the threshold. The threshold can be an absolute number of reads, a percentage of the total number of reads, or a percentage of the total number of SMN1 and SMN2 reads. The threshold can be a percentage of the number of SMN1 and SMN2 reads mapped to the branch node 508a and any associated branch nodes, such as the branch node 508b illustrated in FIG. 5A. As another example, determining the SMA status of the subject can include determining a number of sequence reads mapped to two or more branch nodes, such as the branch nodes 508a, 508b, representing sequence differences between the SMN1 reference sequence and the SMN2 reference sequence. The branch nodes 508a, 508b can represent the single-base difference between SMN1 and SMN2 that affects splicing, which can be used to determine the SMA affected and unaffected status of the subject.

In one embodiment, the branch node can represent a functionally-significant variant of SMN1. Determining the SMA status of the subject can include determining a number of sequence reads mapped to the branch node representing a functionally-significant variant of SMN1. The SMA status of the subject can be determined to be the affected status or the carrier status if the number of sequence reads mapped to the branch node representing the functionally-significant variant is above a threshold. The threshold can be an absolute number of reads, a percentage of the total number of reads, a percentage of the total number of SMN1 and SMN2 reads, or a percentage of the number of SMN1 and SMN2 reads mapped to the branch node and/or any associated branch nodes. Thus, the method 600 can be used to detect known but rare functionally-significant variants in SMN1, helping identify additional individuals who are affected.

In another embodiment, determining the SMA status of the subject includes determining the SMN1 copy number. The computing system can determine the SMN1 copy number by first determining a number of sequence reads mapped to a first branch node representing a first subsequence of the SMN1 reference sequence, such as a cytosine base at position 873 in exon 7 of the SMN1 reference sequence. The first branch node is also referred to herein as a functional site. The computing system can determine a number of sequence reads mapped to a second branch node representing a second subsequence of the SMN1 reference sequence. The second branch node can be referred to herein as a linked site. The first subsequence and the second subsequence can have a high co-occurrence probability. Table 1 shows exemplary functional site and linked site(s) sequences of SMN1.

TABLE 1
Strongly linked variants.

| | Chromosome | | Reference | Alternative |
|---|---|---|---|---|
| Chromosome | Position | Site Classification | Sequence | Sequence |
| chr5 | 70247773 | Functional | C | T |
| chr5 | 70246793 | Linked | G | A |
| chr5 | 70247290 | Linked | T | C |
| chr5 | 70247724 | Linked | G | A |
| chr5 | 70247921 | Linked | A | G |
| chr5 | 70248036 | Linked | A | G |

Thus, the SMN1 copy number can be determined based on the number of sequence reads mapped to the second non-branch node representing the linked site and/or the number of sequence reads mapped to the first branch node representing the functional site. For example, the SMN1 copy number can be determined to be zero if the number of sequence reads mapped to the first branch node representing the functional site is equals to the threshold, such as zero, or below the threshold. The SMN1 copy number can be determined to be one or more if the number of sequence reads mapped to the first branch node representing the functional site is below the first threshold. The SMN1 copy number can be determined to be one if the number of sequence reads mapped to the second branch node representing the linked site is below a second threshold. The SMN1 copy number can be determined to be two (or more) if the number of sequence reads mapped to the second branch node representing the linked site is above the second threshold. The threshold can be an absolute number of reads, a percentage of the total number of reads, a percentage of the total number of SMN1 and SMN2 reads, a percentage of the number of SMN1 and SMN2 reads mapped to the branch node representing the functional site, or a percentage of the number of SMN1 and SMN2 reads mapped to the non-branch node representing the linked site.

In another embodiment, known variants in SMN1 can be used to identify specific haplotypes, which can be used to detect a silent-carrier haplotype that has two copies of SMN1 on a single chromosome, leading to improved carrier status testing. For example, the computing system can determine the SMA status of the subject by determining a number of sequence reads mapped to a branch node representing a variant of SMN1; and determining the spinal muscular atrophy (SMA) status of the subject as the silent-carrier haplotype if the number of sequence reads mapped to a branch node representing the variant of SMN1 is above a threshold. In one embodiment, a branch node can represent a carrier tagging variants of SMN1, whose presence indicates a high probability of the carrier status. Determining the SMA status of the subject can include determining a number of sequence reads mapped to the branch node representing a carrier tagging variant. Table 2 shows exemplary carrier tagging variants.

TABLE 2
Carrier Tagging Variants

| | Chromosome | Reference | Alternative |
|---|---|---|---|
| Chromosome | Position | Sequence | Sequence |
| chr5 | 70243571 | G | A |
| chr5 | 70246957 | A | G |
| chr5 | 70247901 | T | G |
| chr5 | 70248471 | CTA | C |

### Computing Device

FIG. 7 depicts a general architecture of an example computing device 700 configured to learn a demographic model and generate a prediction result using the model. The general architecture of the computing device 700 depicted in FIG. 7 includes an arrangement of computer hardware and software components. The computing device 700 may include many more (or fewer) elements than those shown in FIG. 7. It is not necessary, however, that all of these generally conventional elements be shown provide an enabling disclosure. As illustrated, the computing device 700 includes a processing unit 740, a network interface 745, a computer-readable medium drive 750, an input/output device interface 755, a display 760, and an input device 765, all of which may communicate with one another by way of a communication bus. The network interface 745 may provide connectivity to one or more networks or computing systems. The processing unit 740 may thus receive information and instructions from other computing systems or services via a network. The processing unit 740 may also communicate to and from memory 770 and further provide output information for an optional display 760 via the input/output device interface 755. The input/output device interface 755 may also accept input from the optional input device 765, such as a keyboard, mouse, digital pen, microphone, touch screen, gesture recognition system, voice recognition system, gamepad, accelerometer, gyroscope, or other input device.

The memory 770 may contain computer program instructions (grouped as modules or components in some embodiments) that the processing unit 740 executes to implement one or more embodiments. The memory 770 generally includes RAM, ROM and/or other persistent, auxiliary or non-transitory computer-readable media. The memory 770 may store an operating system 772 that provides computer program instructions for use by the processing unit 740 in the general administration and operation of the computing device 700. The memory 770 may further include computer program instructions and other information for implementing aspects of the present disclosure. For example, in one embodiment, the memory 770 includes a spinal muscular atrophy status determination module 774 that determines the affected or carrier status for spinal muscular atrophy. In addition, memory 770 may include or communicate with data store 780 and/or one or more other data stores that store data for analysis or analysis results.

### EXAMPLES

Some aspects of the embodiments discussed herein are disclosed in further detail in the following one or more examples, which are not in any way intended to limit the scope of the present disclosure.

### Example 1

### Determining SMN1- and SMN2-Specific Copy Numbers

This example describes using quasi-alleles-supporting read counts at multiple positions to determine SMN1- and SMN2-specific copy numbers.

FIG. 8 is an exemplary plot of the sum of read counts supporting SMN2 vs. the sum of read counts supporting SMN1, which can be used to determine SMN1- and SMN2-specific copy numbers. Over 1300 samples were analyzed with whole genome sequencing using Illumina sequencers. Sequencing data from each sample were processed and analyzed by aligning the sequencing data to an SMN2-depleted reference genome as described with reference to FIG. 1 and determining the affected and carrier status of spinal muscular atrophy as described with reference to FIG. 3. Each point in FIG. 8 corresponds to a sample. The x value is the sum, over the “almost always het” sites, of the number of reads supporting the SMN1 reference “allele” at each position. The y value is the sum, over the same sites, of the number of reads supporting the SMN2 reference “allele” at each position. Ovals were added to highlight clusters of samples identified. The slope of each oval was matched to the slope of a line going through the origin and the center of the cluster identified by the oval. Clusters appear to correspond to copy numbers of SMN1 and SMN2. The dashed line is a determination at the boundary between carriers and non-carriers.

The following is a list of positions in the SMN1 gene (on chromosome 5, using the hg19 human reference genome sequence) that was used to generate FIGS. 8: 70244142, 70245876, 70246019, 70246156, 70246320, 70246793, 70246864, 70246919, 70247219, 70247290, 70247724, 70247773, 70247921, and 70248036. The bases at these positions in SMN1 differ from the analogous positions in SMN2, thus resulting in quasi-heterozygous calls in nearly all the samples analyzed.

Altogether, these data demonstrate that at least fourteen positions in the SMN1 gene that are quasi-heterozygous in virtually all samples. Counts of reads that support the SMN1 quasi-alleles at these positions can be used to infer the number of intact SMN1 copies present in the sample. Similarly, the SMN2 copy number can be determined.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system for determining the status of paralogs in a subject comprising:
non-transitory memory configured to store executable instructions; and
a hardware processor programmed by the executable instructions to perform a method comprising:
gathering nucleotide sequence data from a subject comprising first paralog sequence data and second paralog sequence data;
aligning the nucleotide sequence data to a first reference sequence of the first paralog to determine a plurality of alignments;
determining sequence differences between the first paralog sequence data and the reference sequence based on the alignments;
determining a first paralog copy number based on (i) the sequence differences and (ii) a plurality of sequence differences between the reference sequence of the first paralog sequence data and a reference sequence of the second paralog; and
determining a paralog status of the subject based on the first paralog copy number.

2. The system of claim 1, wherein gathering the nucleotide sequence data comprises receiving whole genome sequence data of the subject.

3. The system of claim 1, wherein the first paralog sequence data comprises Survival of Motor Neuron 1 (SMN1), DUX4, RPS17, or CYP2D6/7 gene data.

4. The system of claim 1, wherein aligning the nucleotide sequence data comprises aligning the first paralog sequence data to the first reference sequence and the second paralog sequence data to the first reference sequence.

5. The system of claim 1, wherein determining the sequence differences comprises determining at least one sequence difference between (1) a first sequence read of the sequence data aligned to the reference sequence of the first paralog, and (2) a corresponding subsequence of the reference sequence of the first paralog.

6. The system of claim 1, wherein the paralog status of the subject comprises a copy number of the first paralog or a disease status based on the plurality of sequence differences.

7. A system for diagnosing spinal muscular atrophy (SMA) in a subject comprising:
non-transitory memory configured to store executable instructions; and
a hardware processor programmed by the executable instructions to perform a method comprising:
aligning survival of motor neuron 1 (SMN1) sequence data and survival of motor neuron 2 (SMN2) sequence data from a subject to a SMN1 reference sequence to generate alignments;
determining sequence differences between the SMN1 sequence data and the SMN2 sequence data to the SMN1 reference sequence based on alignments;
determining a SMN1 copy number based on (i) the plurality of sequence differences and (ii) a plurality of differences between the reference SMN1 sequence and a SMN2 reference sequence; and
determining an SMA status of the subject based on the SMN1 copy number.

8. The system of claim 7, wherein aligning the SMN1 sequence data and SMN2 sequence data to the reference SMN1 sequence comprises aligning sequence data comprising the SMN1 sequence data and the SMN2 sequence data to the SMN1 reference sequence and the SMN2 reference sequence.

9. The system of claim 8, wherein aligning the SMN1 sequence data and SMN2 sequence data to the reference SMN1 sequence further comprises:
selecting sequence data aligned to the SMN1 reference sequence or the SMN2 reference sequence; and
aligning the sequence data selected to the SMN1 reference sequence.

10. The system of claim 7, wherein determining the sequence differences comprises determining at least one sequence difference between a first sequence read of the sequence data of SMN1 and a corresponding sequence of the SMN1 reference sequence.

11. The system of claim 7, wherein the hardware processor is further programmed by the executable instructions to:
generate quasi-variant base calls based on the differences between alignments of the SMN1 sequence data and the SMN2 sequence data to the SMN1 reference sequence; and
determine the existence of known variants in the SMN1 sequence data and the SMN2 sequence data based on the quasi-variant calls.

12. The system of claim 11, wherein the hardware processor is further programmed by the executable instructions to determine novel variants in the SMN1 sequence data and the SMN2 sequence data based on the quasi-variant calls.

13. A system for distinguishing paralogs comprising:
non-transitory memory configured to store executable instructions and a data structure representing a plurality of paths comprising a plurality of branch nodes and a plurality of non-branch nodes, wherein the plurality of paths represents a reference sequence of a first paralog, sequence differences between the reference sequence of the first paralog and a reference sequence of a second paralog, variants of the first paralog, and variants of the second paralog; and
a hardware processor programmed by the executable instructions to perform a method comprising:
receiving sequence data of the first paralog and the second paralog of a subject;
mapping the sequence data to at least one branch node or non-branch node associated with a path of the plurality of paths;
determining a number of sequence reads of the sequence data mapped to each branch node or non-branch node; and
determining a paralog status of the subject based on the number of sequence reads mapped to each branch node or non-branch node.

14. The system of claim 13, wherein the first paralog comprises Survival of Motor Neuron 1 (SMN1), DUX4, RPS17, or CYP2D6/7 gene sequences.

15. The system of claim 13, wherein the sequence data of the first paralog and the second paralog comprise a plurality of sequence reads of Survival of Motor Neuron 1 (SMN2) and Survival of Motor Neuron 2 (SMN2) of a subject.

16. The system of claim 13, wherein receiving the sequence data comprises receiving whole genome sequence data of the subject.

17. The system of claim 13, wherein mapping the sequence data to at least one branch node or non-branch node of the path comprises determining an alignment of a sequence read of the sequence data to the at least one branch node or non-branch node of the path based on the sequence read and a sequence represented by the branch node or non-branch node.

18. The system of claim 13, wherein determining the number of sequence reads of the sequence data mapped to each branch node or non-branch node comprises incrementing a count number associated with a branch node or non-branch node when a sequence read is mapped to the branch node or non-branch node.

19. The system of claim 13, wherein the paralog status of the subject comprises a copy number of the first paralog or a disease status associated with the copy number of the first paralog.

20. The system of claim 13, wherein the copy number is determined based on two or more nodes with a high probability of occurring together.

21. A system for spinal muscular atrophy diagnosis comprising:
non-transitory memory configured to store executable instructions and a data structure representing a plurality of paths comprising a plurality of branch nodes and a plurality of non-branch nodes, wherein the plurality of paths represents a survival of motor neuron 1 (SMN1) reference sequence, sequence differences between the SMN1 reference sequence and a survival of motor neuron 2 (SMN2) reference sequence, variants of SMN1, and variants of SMN2; and
a hardware processor programmed by the executable instructions to perform a method comprising:
receiving a plurality of sequence reads of SMN1 or SMN2 of a subject;
mapping each of the plurality of sequence reads to at least one branch node or non-branch node of a path of the plurality of paths;
determining a number of sequence reads mapped to each of the plurality of branch nodes; and
determining a spinal muscular atrophy (SMA) status of the subject based on the number of sequence reads mapped to each of the plurality of branch nodes.

22. The system of claim 21, wherein determining the SMA status of the subject comprises:
determining a number of sequence reads mapped to a branch node representing a sequence difference between the SMN1 reference sequence and the SMN2 reference sequence; and
determining the SMA status of the subject as:
the affected status if the number of sequence reads mapped to the branch node representing the SMN1 reference sequence is below a threshold, and
a carrier status or an unaffected status otherwise.

23. The system of claim 22, wherein the branch node represents a cytosine base at position 873 in exon 7 of the SMN1 reference sequence.

24. The system of claim 21, wherein determining the SMA status of the subject comprises:
determining a number of sequence reads mapped to a branch node representing a functionally-significant variant of SMN1; and
determining the SMA status of the subject as:
an affected status or a carrier status if the number of sequence reads mapped to the branch node representing the functionally-significant variant is above a threshold.

25. The system of claim 21, wherein determining the SMA status of the subject comprises determining the SMN1 copy number.

26. The system of claim 25, wherein determining the SMN1 copy number comprises determining the SMN1 copy number based on the number of sequence reads mapped to a branch node.

27. The system of claim 25, wherein determining the SMN1 copy number comprises determining a number of sequence reads mapped to a first branch node representing a first subsequence of the SMN1 reference sequence.

28. The system of claim 25, wherein determining the SMA status of the subject comprises determining a number of sequence reads mapped to a branch node representing a variant of SMN1.

29. The system of claim 21, wherein the hardware processor is further programmed by the executable instructions to generate the data structure representing the plurality of paths.

30. The system of claim 21, wherein the hardware processor is further programmed by the executable instructions to graphically display the plurality of branch nodes and the plurality of non-branch nodes as a graph.

31. The system of claim 21, wherein a path of the plurality of paths comprising one or more non-branch nodes and one or more branch nodes represents the SMN1 reference sequence.

32. The system of claim 21, wherein two branch nodes represent a difference between the SMN1 reference sequence and the SMN2 reference sequence, a difference between the SMN1 reference sequence and a variant of SMN1, a difference between the SMN2 reference sequence and a variant of SMN2, or any combination thereof.

33. The system of claim 21, wherein one non-branch node represents an insertion of at least one nucleotide into the SMN1 reference sequence or a deletion of at least one nucleotide from the SMN1 reference sequence.
